(19) United States
(12) Patent Application Publication
(10) Pub. No.: US 2024/0410853 A1
(43) Pub. Date: Dec. 12, 2024

(54) METHOD FOR DETERMINING AN AMOUNT OF CHEMICALLY BOUND CARBON DIOXIDE AND DEVICE FOR DETERMINING SAID AMOUNT

(71) Applicant: HEMMERSBACH GMBH & CO. KG, Nürnberg (DE)

(72) Inventor: Dirk PAESSLER, Fürth (DE)

(21) Appl. No.: 18/700,944

(22) PCT Filed: Oct. 24, 2022
(86) PCT No.: PCT/EP2022/079669

§ 371 Date: Apr. 12, 2024

(30) Foreign Application Priority Data
Oct. 25, 2021 (DE) 10 2021 127 692.6

(51) Int. Cl. G01N 27/333; G01N 33/24

(52) U.S. Cl. CPC G01N 27/333; G01N 33/24

(57) ABSTRACT

A method for determining an amount of carbon dioxide which, with the aid of a reaction agent located on the ground, is chemically bound within a time interval. —reading in at least one reaction agent parameter relating to the reaction agent; —measuring a measurement value for the conductivity of the ground and/or for an individual cation concentration of at least one cation in the ground by at least one sensor installed in or on the ground; and —determining the amount of the bound carbon dioxide in accordance with both the reaction agent parameter and the measurement value or the measurement values.

The invention relates to a method for determining an amount of carbon dioxide chemically bound within a time interval with the aid of a reaction agent on the ground. In addition, the invention relates to a device for determining this amount.

Carbon dioxide emission results in higher carbon dioxide concentrations in the atmosphere and therefore in a long term temperature increase due to the greenhouse effect. In a moderate scenario, in which it is presumed that the emission of carbon dioxide will sink again from approximately the middle of the century due to climate protection measures, in current model calculations a temperature increase of approximately 2.7° C. by the end of the century is presumed. In contrast, if a reduction of the carbon dioxide emission does not succeed or only succeeds at a significantly later point in time, significantly higher temperature increases are also possible. Corresponding temperature increases also result, in addition to other problems, in a significant rise of sea level, which can be significantly above a meter in the long term.

It is essential to reduce the use of fossil fuels and the carbon dioxide emission connected thereto to restrict the temperature increase and avoid or reduce its negative effects. However, since it is very challenging to achieve a carbon dioxide-neutral economy solely by reducing the carbon dioxide emission and since a reduction of the carbon dioxide already present in the atmosphere solely by natural processes is very protracted, it is expedient to use measures for removing the carbon dioxide from the air and binding it.

One promising approach for this purpose is improving or strengthening natural weathering processes in which specific types of rock are decomposed in the presence of carbon dioxide and water, wherein parts of the carbon dioxide participating in the reaction are chemically bound by forming carbonates. Since such weathering processes run on the rock surface, they are typically very slow. However, it is known that significantly accelerated weathering and thus carbon dioxide binding can be achieved by a significant enlargement of the surface, for example, in that the rock is initially ground to fine dust. This is also referred to as “enhanced weathering”.

For example, minerals from the olivine group can bind between 0.5 t and 1.5 t carbon dioxide per ton. Other types of rock, for example, basalt, dunite, kimberlite, or wollastonite are also suitable for this purpose. The use of accelerated weathering for carbon dioxide binding is discussed, for example, in document WO 2020/263910 A1.

Since the mentioned minerals or types of rock are partially permissible fertilizers, in principle very large areas would be ready for such accelerated weathering, so that a large amount of carbon dioxide could be bound and therefore a significant contribution could be provided to reducing climate change.

However, to achieve this, this land use has to be attractive for farmers and landowners. This could be achieved in that due to the negative emission by the carbon dioxide binding by farmers and landowners, emission certificates can be sold to compensate for carbon dioxide emissions. For this purpose, however, it would be necessary to quantify the amount of bound carbon dioxide. However, since significantly different weathering speeds can result in the case of accelerated weathering in an open field than in the laboratory, approaches are required for this purpose of quantifying the actual amount of bound carbon dioxide.

If, as in the above-mentioned document, it is presumed that the bound carbon dioxide is separated again from the weathered rock, for example, due to the effective temperature, it would be possible to measure the amount of bound carbon dioxide directly. However, this would be very complex since for this purpose the rock would have to be collected again from large fields or open areas and complexly postprocessed. Moreover, the carbon dioxide separated from the weathered rock would then have to be stored in another way. In contrast, if it is presumed that the weathered rock is directly used for the long-term binding of carbon dioxide, such a direct measurement is not possible.

The invention is therefore based on the object of determining the amount of carbon dioxide chemically bound by a reaction agent on the ground, for example, by the above-mentioned types of rock or mineral, wherein this is to take place in particular without breaking this chemical bond.

The object is achieved according to the invention by a method for determining an amount of carbon dioxide chemically bound within a time interval with the aid of a reaction agent on the ground, which comprises the following steps:
reading in at least one reaction agent parameter relating to the reaction agent,
acquiring a respective measured value for the conductivity of the ground and/or for a respective cation concentration of at least one cation in the ground by at least one sensor attached in or on the ground, and
determining the amount of the bound carbon dioxide as a function of both the reaction agent parameter and the measured value or the measured values.

It has been recognized in the scope of the invention that the amount of the bound carbon dioxide can be determined with good accuracy if, on the one hand, the properties of the reaction agent, thus, for example, its grain size and/or content of specific minerals, is taken into consideration by the reaction agent parameter and, on the other hand, at least one measured value is taken into consideration, which relates to the specific conditions of the weathering on location.

The reaction agent parameter can describe, for example, the amount of the bound carbon dioxide for a specific amount of reaction agent or the time derivative thereof, thus a binding rate. Such a reaction agent parameter can be determined, for example, in that weathering takes place under laboratory conditions for samples of the reaction agent, by which the precise amount of the absorbed carbon dioxide or the absorption rate under these conditions can be determined. However, it is also possible that at least one other item of information, such as a grain size and/or a mineral content of the reaction agent, is read in as the reaction agent parameter, wherein, for example, characteristic maps or known mathematical relationships, which are determined by regression, for example, can be used to determine therefrom the amount of carbon dioxide that can be bound per amount of reaction agent, a binding rate, or the like.

As will be explained later in detail, the amount of carbon dioxide bound by a reaction agent within a time interval or the binding rate is dependent on a large number of factors. However, it has been recognized in the scope of the invention that by taking into consideration the conductivity of the ground or the cation concentration of at least one cation, a significant improvement of the accuracy of the determined amount of the bound carbon dioxide can already be achieved in relation to exclusively considering the reaction agent parameter. Depending on the application, it can therefore already be sufficient to take into consideration one or more of the mentioned measured values in the determination of the bound carbon dioxide. On the other hand, it can be expedient in other applications to further improve the accuracy of the determination in that additional items of information are taken into consideration, as will be explained later.

One essential advantage of the method according to the invention is that due to a sensory acquisition of measured values directly at the reaction location or weathering location, a time resolution of the measured value acquisition and thus the determination of the carbon dioxide binding can be achieved, which cannot be achieved with acceptable expenditure by other approaches for taking into consideration local influences on the weathering, for example, by taking and studying soil samples. For example, by using wirelessly read sensors, it is possible to update the measured values several times a day or even several times an hour without problems, so that the local reaction environment can be mapped significantly better than if, for example, soil samples were only taken weekly or monthly.

For the measured value or the measured values, in particular time curves can be acquired, by which in particular a time curve of the amount of the carbon dioxide bound per time interval can be determined. The amount of carbon dioxide bound in the entire time interval can then be determined by a time integral or summation over the measurement intervals in which measurements take place.

Suitable sensors for determining the above-mentioned measured values or the sensor data discussed later, which can additionally be taken into consideration, are known as such from the field of intelligent agriculture. For example, the Internet site “https://www.dragino.com/products/lora-lorawan-end-node/item/159-Ise01.html” describes a sensor for monitoring the ground moisture, temperature, and conductivity. The Internet site “https://teralytic.com/how-it-works.html” discloses, for example, measurements of salinity, pH value, and concentrations of specific ions. It is possible to adapt the selectivity for specific ions or cations, for example, by selecting a suitable ion-selective electrode.

The amount of the bound carbon dioxide can be determined as a function of sensor data of the sensor and/or at least one further sensor, wherein the sensor data relate to a vertical water flow in the ground and/or an amount of precipitation and/or a moisture in the ground and/or an alkalinity and/or a nutrient content of the ground and/or a partial pressure of carbon dioxide in the air above the ground. By using all or also only parts of the mentioned sensor data, the accuracy of the determination of the amount of the bound carbon dioxide can be further improved.

The sensor data can be acquired in this case locally by a sensor attached in the area of the ground or within the ground. For example, for the amount of precipitation, it can be advantageous, however, to use a satellite instead of the sensor, wherein in this case the assignment of the sensor data to the region for which the amount of the bound carbon dioxide is to be determined can take place, for example, in that the location of the region is stored or, as will be explained later, is determined on the basis of GPS data, for example. The partial pressure of carbon dioxide can in particular be determined close to the ground, for example, in that a measurement module, which is partially inserted into the ground, for example, has a suitable sensor in its section protruding above the ground.

The vertical water flow in the ground and/or an amount of precipitation can be measured in particular by a lysimeter. For example, a lysimeter based on vertically spaced-apart moisture sensors can be used, by which low-maintenance operation and electronic readout are particularly easily implementable.

A direct measurement of the alkalinity can be relatively complex. For this purpose, for example, a sample of the soil can be taken and studied in an automated manner. It can therefore be advantageous to instead determine or estimate the alkalinity on the basis of further sensor data or measured values, for example, in that a change of the pH value expected on the basis of other parameters is compared to an actual pH value change. For this purpose, it can be expedient to provide additional data from external sources, which describe, for example, a type of the planting, a specific ground property, or the like. These additional data can either be transferred to the location of the sensor or the sensors or the calculation can take place, for example, on a server or another data processing device spaced apart from the server.

Sensor data which relate to the nutrient content of the ground can relate in particular to the concentration of nitrogen, potassium, or phosphorus compounds or ions in the soil.

The amount of the bound carbon dioxide can be determined as a function of sensor data of the sensor and/or the further sensor and/or at least one further sensor, wherein the sensor data relate to a pH value and/or a temperature of the soil. The pH value and the temperature of the ground can also influence the binding of carbon dioxide or the occurrence of carboxylic acid in the scope of the binding process can be recognized on the basis of the pH value.

In particular, several or even all of the various above-mentioned sensor data can be taken into consideration in the determination of the amount of the bound carbon dioxide. By evaluating a broad spectrum of measured parameters, which influence the weathering and/or are influenced thereby, a high accuracy can be achieved for the determination of the amount of the bound carbon dioxide, wherein a high time resolution can be achieved on the basis of the sensory acquisition and moreover a high location resolution can be achieved upon use of multiple spaced-apart sensors.

The reaction agent parameter can specify a base value for the amount of the bound carbon dioxide or for the time derivative of this amount, which is multiplied in the course of the calculation with at least one correction factor, wherein at least one of the correction factors is dependent on the measured value or at least one of the measured values and/or on at least parts of the sensor data. It has been shown that with such a calculation, corrections due to different influences are particularly well separable. At least one of the correction factors can be dependent both on at least one of the mentioned variables and on at least one of the items of prior information which will be explained later. Individual correction factors can also be exclusively dependent on at least one of the items of prior information.

As an example, a weathering rate Rᵣₑₐₗ at the time t, which can correspond to the time derivative of the amount of the bound carbon dioxide or can be proportional thereto, can be calculated as the product of the base value R_{base} for this time derivative and 6 correction factors:

Rᵣₑₐₗ(t)=R_{base}(t)*f_{conduct}(t)*f_{ground}(t)*fₑₙᵥᵢᵣₒₙₘₑₙₜ(t)*f_{liquid}(t)*f_{pH}(t)*fₐₗₖ (t)

The base value R_{base} can be determined as explained above, for example, under laboratory conditions. The various correction factors f can each be dependent on at least one of the measured values and/or on sensor data and/or on items of prior information which will be explained later. To determine the respective correction factor f from the respective input variables, for example, experimental series can be carried out in order to determine such a relationship, for example, as a lookup table or by a regression analysis or also by machine learning.

The correction factor f_{conduct} can be exclusively dependent, for example, on the measured value for the conductivity of the ground. The correction factor f_{ground} can relate to further properties of the ground and in particular be dependent on the at least one measured value for the cation concentration and/or on items of prior information, which relate in particular to a porosity and/or a soil type of the ground and/or fungi and/or bacteria present in the ground and/or a planting of the ground.

The correction factor fₑₙᵥᵢᵣₒₙₘₑₙₜ can relate to environmental influences and in particular be dependent on a temperature and/or an amount of precipitation measured as part of the sensor data or items of prior information relating to these variables. The correction factor f_{pH} is determined from a pH value of the ground acquired in particular as sensor data and the correction factor fₐₗₖ is determined from an alkalinity, which can be acquired as sensor data or provided as prior information. The use of the six above-mentioned correction factors is only one possible example of an advantageous correction factor combination. It is also possible, for example, not to take into consideration one or more of the mentioned correction factors or to use further correction factors.

The individual correction factors f typically vary around the value 1 and each express a certain increase or reduction of the time derivative of the amount of the bound carbon dioxide due to the influencing factors, on which they are dependent. It is not necessary to use all mentioned correction factors f, however, the consideration of a large number of influencing factors can improve the accuracy of the determined weathering rate Rᵣₑₐₗ overall.

The amount of the carbon dioxide chemically bound within a time interval with the aid of a reaction agent on the ground can be determined analytically by integration of the weathering rate Rᵣₑₐₗ over time. Since the various influencing variables, thus in particular the measured value or the measured values and the sensor data, are typically acquired in a time-discrete manner in any case, however, such an integral can be approximately calculated by summation over the measurement intervals. In this case, it is not necessary for the same measurement interval to be used for all influencing factors.

The concentration of sodium ions and/or of calcium ions and/or of magnesium ions can be acquired as the cation concentration. In particular, the concentration of that cation or those cations can be determined as the cation concentration, which were parts of the reaction agent before the weathering or reaction.

The weathering reaction will be explained hereinafter on the basis of the example of the weathering of forsterite (Mg₂SiO₄). The following reaction equation can be specified for this purpose:

Mg₂SiO₄+4CO₂+4H₂O+↔2Mg²⁺+4HCO₃⁻+↔2MgCO₃+SiO₂+2CO₂+4H₂O

As can be seen from this reaction equation, half of the carbon dioxide molecules used are bound as magnesium carbonate by the weathering of the forsterite in the presence of carbon dioxide and water. Dissolved magnesium cations are present in an intermediate step of this reaction, so that the concentration of these cations correlates with the weathering rate.

The respective measured value and/or the sensor data and/or at least one intermediate result determined from the measured value and/or the sensor data can be transmitted wirelessly from a measurement position, at which the respective sensor or the respective sensors is or are arranged, to a processing device, which carries out the determination of the amount of the bound carbon dioxide. The communication can be implemented, for example, by a mobile wireless protocol, e.g., 3G, LTE, 5G, Edge, etc., or by a Wide Area Network, in particular a Long Range Wide Area Network (LoRaWAN, such as Zero-G).

The use of a processing device spaced apart from the respective measurement position is expedient in particular if, in the determination of the amount of the bound carbon dioxide, measured values or sensor data from multiple different measurement positions, for example, from various fields used for agriculture, which each have a separate sensor system, are to be collected and evaluated jointly. This can be expedient, for example, in order to achieve not only a high time resolution, but also a high spatial resolution for the measured values or the sensor data and thus to further improve the accuracy of the determination.

The processing device can be, for example, a server, a cloud-based solution, or also a workplace computer. The use of a separate processing device can also be expedient if, for example, items of prior information are to be used in the course of determining the amount of bound carbon dioxide, which are stored in this processing device or are to be acquired from external sources, for example, a laboratory which studies soil samples, for example, via the Internet.

The processing device itself or a further device supplied thereby with items of information with respect to the amount of the bound carbon dioxide can provide these items of information, for example, via a web interface, an app, or the like to the user. In this case, brief delays of significantly less than an hour, in particular of less than 10 minutes, between the acquisition of the measured values or the sensor data and the provision of an updated amount of bound carbon dioxide can be achieved, so that farmers providing land or similar method participants can be informed just in time, for example.

In the method according to the invention, the measured value or the measured values and/or at least parts of the sensor data can be acquired several times a day or several times per hour and the amount of the bound carbon dioxide can be determined as a function thereof. Due to a high time resolution of the measurement and determination, a high accuracy is achieved, which can also recognize and take into consideration short-term changes of the carbon dioxide absorption, for example, due to brief precipitation. Such a high time resolution of the measurement data acquisition and evaluation would not be implementable with acceptable expenditure, for example, upon a determination of the amount of the bound carbon dioxide exclusively on the basis of taking soil samples.

The measured value or at least one of the measured values and/or the sensor data can be acquired at multiple measurement positions by a respective sensor, wherein the measurement positions are spaced apart from one another in particular by less than 500 m or less than 200 m. For example, at least for parts of the measured values or sensor data, a separate sensor or a separate measurement module which comprises multiple sensors can be provided per field. Therefore, for example, a separate measurement data acquisition can take place per half hectare or per hectare. This is not only advantageous to enable an individual calculation even with relatively small fields of various farmers or in similar situations, it also overall enables an improvement of the amount acquisition, since, for example, local differences with respect to the planting, soil quality, etc. can be taken into consideration with good accuracy.

A soil sample can be taken from the ground, in particular in the area of a respective measurement position, at which the respective sensor or the respective sensors is or are arranged, before acquisition of the respective measured value and/or the sensor data, which soil sample is studied, in particular spaced apart from the removal location in a laboratory, in order to determine an alkalinity and/or a porosity and/or a soil type of the ground and/or fungi and/or bacteria present in the ground as items of prior information, wherein the amount of the bound carbon dioxide is determined as a function of the prior information. As the soil type it can be determined, for example, whether it is sandy or loamy soil or the like.

As already explained, it is typically not possible to take soil samples with the same frequency as a measurement of measured values or sensor data by sensors. At the same time, the mentioned parameters can be determined more accurately or at all for the first time with the aid of soil samples and, for example, laboratory studies and thus taken into consideration. It can therefore be advantageous to take soil samples at relatively long time intervals of, for example, several weeks or even months, in order to determine items of information about the mentioned, relatively slowly changing parameters of the ground, while parameters which change faster can be acquired by sensors and taken into consideration.

A respective item of prior information, which relates in particular to an alkalinity and/or a porosity and/or a soil type of the ground and/or fungi and/or bacteria present in the ground and/or a planting of the ground and/or the weather, and/or the respective prior information determined on the basis of the soil sample can be provided separately for multiple areas, wherein in a measurement area, which comprises the sensor and/or the further sensor, a position determination device is additionally used, which provides an item of position information relating to the respective measurement area, wherein the prior information assigned to the measurement area is selected on the basis of the position information, according to which as a function thereof and the at least one measured value acquired in the measurement area and/or of at least parts of the sensor data acquired in the measurement area, the amount of the bound carbon dioxide or an intermediate result, on which the determined amount of the bound carbon dioxide is dependent, is determined. In particular a time derivative or a change over time of the amount can be determined as the intermediate result, from which the amount can be determined by integration or time-discrete summation.

The prior information can be determined by a soil sample or can also be manually input, for example, if it relates to a planting. Additionally or alternatively, for example, an item of information can be used as prior information which can be determined via satellite, by an aircraft-based sensor, or the like.

All sensors for a specific measurement area or at least parts of these sensors can be integrated together with the position determination device in a common measurement station. However, it is also possible that multiple distributed sensors communicate, for example, via a wireless local network, with a central station for the respective measurement area, which has a position determination device or also communicates therewith.

If the amount or the intermediate result is determined for multiple measurement areas, the amounts or the intermediate results can be added up or weighted summation can be carried out, for example, to take into consideration different sizes of the measurement areas.

It can be expedient to determine various intermediate results, for example, different ones of the above-explained correction factors, for areas of different sizes. For example, amounts of precipitation or temperatures can be acquired for relatively large measurement areas and a conductivity of the soil or a cation concentration, a nutrient concentration, or the like and the correction factors resulting therefrom can be determined separately as an intermediate result for multiple sub-areas of this larger measurement area.

The determination of the position information also allows the amount of the bound carbon dioxide to be implemented for multiple separate measurement areas, for example, for various fields, by a common processing device. Optionally, a separate billing or certification of the carbon dioxide binding can nonetheless be possible in this case for the individual measurement areas or subgroups of the measurement areas.

The position determination device can carry out, for example, a satellite-based position determination, for example, based on GPS. Alternatively to using a position determination device, it would also be possible, for example, to provide the measured values or sensor data of sensors of a measurement area via a respective communication device to the processing device and in this case to also acquire an identifier of the communication device, for example, a user number in a wireless network. The assignment of the measured values or sensor data to specific measurement areas can then be carried out on the basis of this identifier and a previously specified assignment of the identifiers to the measurement areas. For this purpose, however, it would be necessary to manually assign the individual communication devices to the individual measurement areas, so that a higher expenditure results and a certain risk of error remains, for example, due to inadvertently confusing communication devices or measurement modules.

In addition to the method according to the invention, the invention relates to a device for determining an amount of carbon dioxide chemically bound within a time interval with the aid of a reaction agent on the ground, comprising at least one sensor for acquiring measured values for the conductivity of the soil and/or for a respective cation concentration of at least one cation in the soil and a processing device, wherein the device is configured to carry out the method according to the invention.

In particular a typical data processing device can be used as the processing device, such as a server, a workplace computer, a cloud-based data processing device, a smartphone, a tablet, or the like, which receives the measurement data and in particular the above-explained sensor data from the sensor or the sensors and retains the reaction agent parameter and optionally the prior information or retrieves them via the Internet from a further device for example. The determination of the amount of the bound carbon dioxide and the data acquisition from the sensor, the optionally used position determination devices, etc., can be implemented, for example, by a program, which is executed by the data processing device.

The device can comprise at least one measurement module, which comprises a housing designed to be inserted into the ground, at least one sensor, a communication device for wirelessly transmitting measured values and/or sensor data acquired by means of the sensor and/or an intermediate result determined from the measured value and/or the sensor data to the processing device and at least one energy supply device for supplying the communication device and the sensor. The measurement module can be in the form of a rod or pole at least in one section in order to enable easy introduction into the ground. It can be inserted, screwed, or hammered into the ground, for example. In the state introduced into the ground, a section which is 15 to 100 cm long, in particular a section of the measurement module which is 30 to 50 cm long can be located within the ground. At least parts of the sensors can be present at various heights of the measurement module, for example, to enable a determination of a vertical liquid flow by way of vertically spaced-apart moisture sensors.

The measurement module can comprise a data processing device, which is formed, for example, by a CPU or a microcontroller having assigned memory. This can control the communication device, read the sensor or the sensors, optionally determine the above-explained intermediate result, or the like. The housing can be water-repellent and/or dirt-repellent or can comprise a receptacle space for at least parts of the electronics, e.g., the communication device, the processing device, and/or the energy supply device, which are water-repellent and/or dirt-repellent. In this case, in particular also infiltration of water from the bottom side is to be prevented by a corresponding housing seal. The housing is to protect the electronic components from all weather or seasonal influences to be expected. The housing is preferably made sufficiently stable that, for example, it can be hammered into the ground or that damage to components is prevented, for example, if the measurement module is driven over by a tractor.

A battery and/or a solar panel can be used as the energy supply device. The communication device can in particular be used for communication via a mobile wireless network or a wide area network. Various possible protocols for this purpose have already been discussed above. An antenna for the communication device can also be provided on or in the housing.

The measurement module can in particular comprise a position determination device, such as a GPS module. To enable a configuration and/or a status check on the measurement module itself without additional components, status lights, such as LEDs, operating elements, such as buttons, and/or a display can be provided on the measurement module. Moreover, the measurement module can comprise a signal device, such as a bright flashing light or an acoustic notification device, such as a loud beeper, which can be activated, for example, by a wireless communication to facilitate finding the measurement module.

Further advantages and details of the invention result from the following exemplary embodiments and the associated drawings. In the schematic figures:

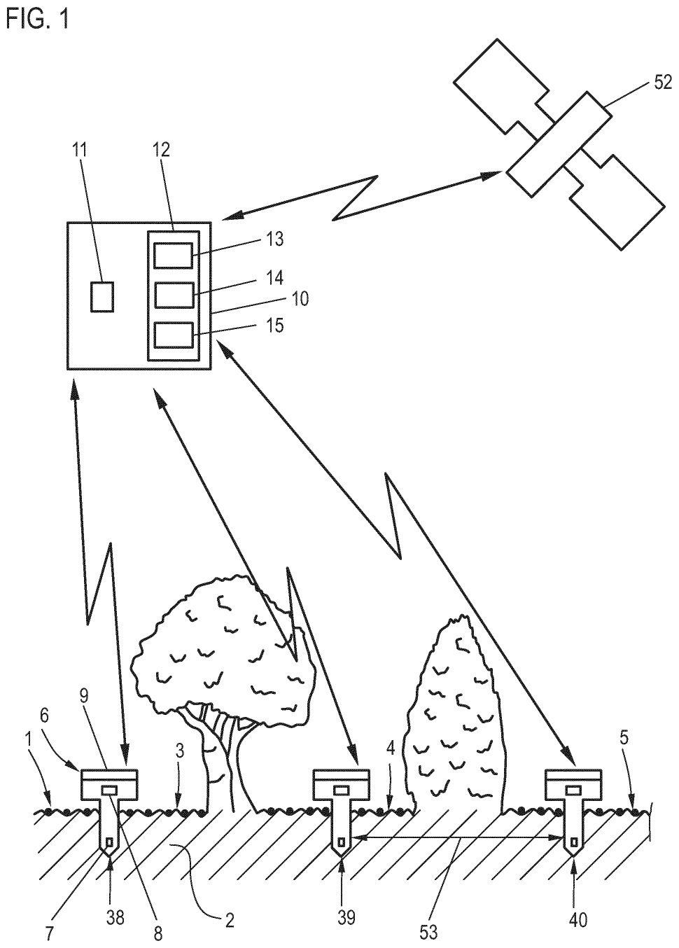

FIG. 1 shows an exemplary embodiment of a device according to the invention for determining an amount of bound carbon dioxide,

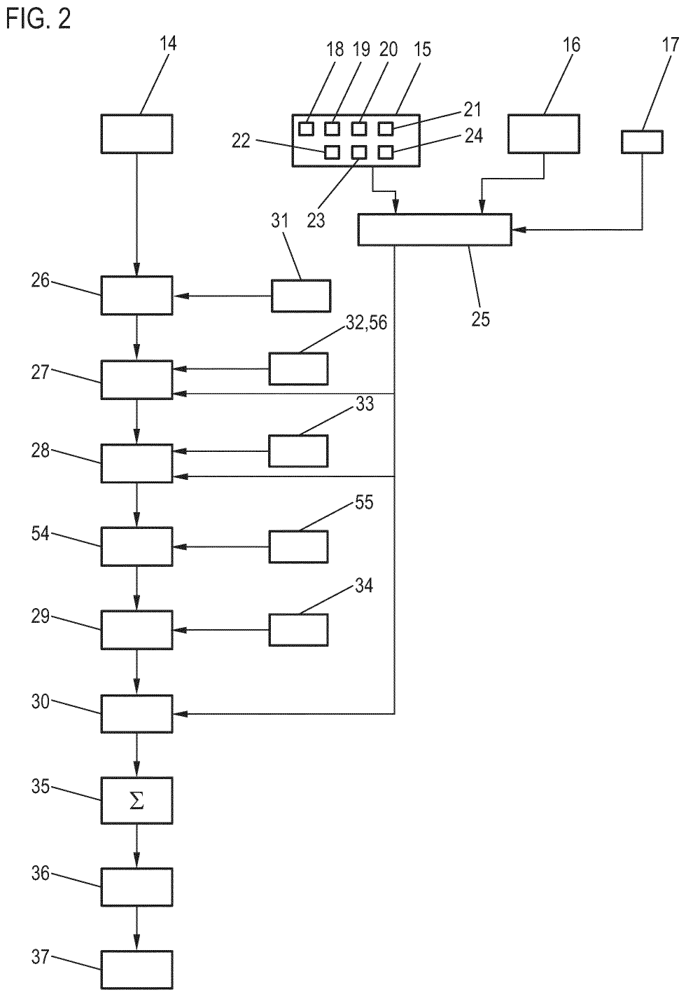

FIG. 2 shows the interaction of relevant algorithms and data structures in the scope of an exemplary embodiment of the method according to the invention for determining the amount of bound carbon dioxide, and

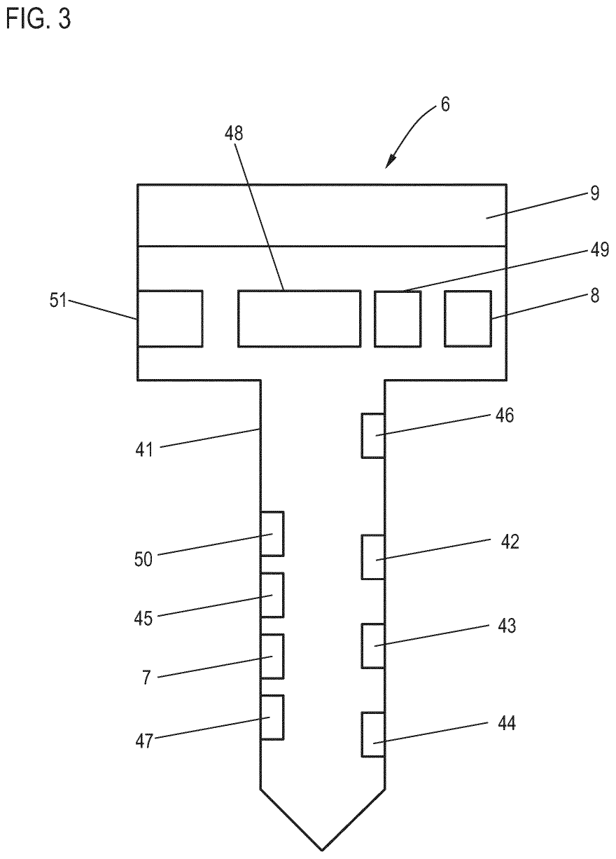

FIG. 3 shows an exemplary embodiment of the measurement module shown in FIG. 1.

FIG. 1 schematically shows a device for determining an amount of carbon dioxide chemically bound within a time interval with the aid of a reaction agent 1 on the ground. To enable a clear representation of the relevant components, the relative dimensions or distances between the objects shown sometimes differ significantly from real size relationships or distances.

Finely ground rock is used as the reaction agent 1, the weathering of which, as already explained in the general part, absorbs carbon dioxide from the air. To provide the largest possible reaction surface, the reaction agent 1 is distributed over a large area on the ground in multiple measurement areas 3, 4, 5, which in the example are separate fields used for agriculture. The amount of the carbon dioxide bound in the respective measurement area 3, 4, 5 is dependent here, on the one hand, on properties of the reaction agent 1 itself and, on the other hand, on a large number of further parameters, such as the soil quality, the weather, and others.

To create an incentive for such carbon dioxide binding, it is highly relevant to quantify the bound amount of carbon dioxide, for example, to be able to take into consideration the binding of carbon dioxide in the context of an emission certificate trade. Attempts could be made to take into consideration relevant influences solely by taking soil samples, which are subsequently studied in a laboratory. However, since short-term influences are also highly relevant for the amount of the bound carbon dioxide, such as rain showers or growth processes of plants, a very frequent study of soil samples would be required, which would result in high costs and thus would not be economically expedient.

In the example shown, a different method is therefore used to determine the amount of the bound carbon dioxide. In this case, sensors 7 on the ground are used, for example, to acquire measured values for the conductivity of the soil, as a function of which the amount of the bound carbon dioxide is determined. The use of local sensors enables relevant measured values to be acquired with high time and spatial resolution, so that a higher resolution can be provided both spatially and with respect to time than would be possible with acceptable expenditure by taking soil samples.

Additionally or alternatively to the acquisition of the conductivity of the ground 2, a cation concentration of at least one cation in the ground 2 can also be determined as measured value or other sensor data, which will also be explained hereinafter with reference to FIG. 2, can additionally also be taken into consideration. However, for reasons of clarity, only precisely the sensor 7 thereof is shown in FIG. 1 per measurement area 3, 4, 5 or per measurement module 6 respectively arranged there. A measurement module 6 having a greater number of sensors will also be explained later with reference to FIG. 3.

One possible procedure for determining the amount of the bound carbon dioxide will be explained hereinafter with additional reference to FIG. 2, which shows the interaction of algorithms and data structures relevant for this purpose. In this case, the calculations are carried out by a processing device 10, which can be a server, for example. A computer program 13 stored in a memory 12 is executed by a processor 11 in order to implement the steps of the method.

In this case, first a reaction agent parameter 14 relating to the reaction agent 1 is read in. This can already be present in the memory 12 of the processing device 10 or can be retrieved from a server or the like, for example, via the Internet. In the example, it is assumed that the reaction agent parameter describes a time derivative of the amount of the carbon dioxide chemically bound by the reaction agent under laboratory conditions. This can be determined, for example, in prior experiments for a specific batch of reaction agents or for a specific reaction agent type.

The reaction agent parameter or the base value for the time derivative of the amount of the bound carbon dioxide is then multiplied by multiple correction factors 26 to 30, as was already explained in the general part of the description.

For simplification it is first to be assumed that exclusively the correction factor 26 is used, which corresponds to the correction factor f_{conduct} discussed in the general part. For this purpose, a measured value 31 for the conductivity of the ground 2 is detected by the respective sensor 7 of the respective measurement module 6 and wirelessly transmitted by a communication device 8 of the respective measurement module 6 to the processing device 10, after which the correction factor 26 is calculated with the aid of a calculation rule provided by the program 13 and the base value is multiplied thereby. The energy supply of the measurement module 6 or in particular the sensor 7 and the communication device 8 is carried out in this case by an energy supply device 9, in the example by a solar cell.

After the base value is multiplied by the correction factor 26, or in the more complex design shown in FIG. 2 by all correction factors 26 to 30, the real weathering rates or the time derivatives of the amount of the bound carbon dioxide for the various measurement areas 3, 4, 5 and for the successive time intervals, for each of which the correction factor 26 potentially differs, are added to one another to determine the amount 36 of the total carbon dioxide bound in the time interval. This can be provided to users or to other processing devices via an output interface 37, for example, via a web interface, for example, to be able to provide emission certificates on the basis of the bound carbon dioxide automatically.

To improve the accuracy of the determination of the amount 36, it is advantageous to additionally use the further correction factors 27 to 30 or at least parts thereof. The correction factor 27 corresponds to the correction factor f_{ground} discussed in the general part. This is dependent, as was already explained there, in particular on a measured value 32 for the cation concentration of at least one cation in the ground 2. In particular, concentrations can be measured for those cations which are initially part of the reaction agent and precipitate as the carbonate by binding of the carbon dioxide.

It can be advantageous to use various sensors for determining the various measured values. A measurement module 6 which comprises various sensors 7, 42 to 47, 50, 51 is schematically shown in FIG. 3 and will be explained in more detail later. The measured value 32 can be provided, for example, by the sensor 45, which measures the cation concentration with the aid of an ion-selective electrode, for example.

The correction factor 27 can additionally be dependent on additional items of information 15, which can be stored beforehand in the memory 12 of the processing device 10 or can be provided via a third device (not shown), for example.

The prior information 15 can be provided multiple times, for example, once for each of the measurement areas 3, 4, 5. To be able to process the prior information in each case jointly with the measured values 31, 32 or sensor data 33, 34, 55 for the respective measurement area 3, 4, 5 or to be able to determine separate amounts of bound carbon dioxide for the individual measurement areas 3, 4, 5, the items of prior information 15 are each assigned to an area 18 or provided separately for each area 18. Moreover, a position determination device 49, such as a GPS sensor, is present in each measurement area 3, 4, 5, which can in particular be integrated in the measurement module 6, as shown in FIG. 3. The position determination device 49 provides an item of position information via the communication device 8 jointly with the measured values 31, 32 or the sensor data 33, 34, so that in each case the item of prior information 15 can be selected by a selection module 25 for joint processing with the measured values 31, 32 or sensor data 33, 34 of a measurement area 3, 4, 5, the area 18 of which corresponds to the respective measurement area 3, 4, 5.

The correction factor 27 is in particular to depict properties of the ground 2, because of which in particular a porosity 20 and/or a soil type 21 of the ground 2 and/or fungi 22 and/or bacteria 23 present in the ground 2 and/or a planting 24 of the ground 2 can be provided as prior information 15 for determining the correction factor 27. These items of prior information 15 can be determined, for example, in that soil samples are studied in the laboratory or they can be manually input, for example, to take into consideration seeds dispensed as the planting 24.

In addition to the dependencies shown, the correction factor 27 can additionally depend on a nutrient content of the ground 2, which can be determined, for example, via a further sensor 50, which is shown in FIG. 3. This sensor can measure, for example, the concentration of sodium, phosphorus, and/or potassium compounds or ions, which are provided by nutrients in the ground.

The correction factor 28 corresponds to the correction factor fₑₙᵥᵢᵣₒₙₘₑₙₜ discussed in the general part. This is in general to take into consideration the weather or especially a temperature and an amount of precipitation. In the example, the temperature can be acquired by the temperature sensor 46 shown in FIG. 3. The amount of precipitation could in principle also be measured locally, however, a sensor 52, which is satellite-based, is used for this purpose in the example.

Since amounts of precipitation for a large number of areas are acquired via such a satellite-based sensor 52, the sensor data 16 provided by the sensor 52 can also be processed by the selection module 25 in order to select suitable partial data with the aid of the position information 17, which are taken into consideration in the determination of the correction factor 28.

Optionally, sensor data which relate to a partial pressure of carbon dioxide in the air above the ground 2 can also be taken into consideration in the determination of the correction factor 28 or for the determination of a further correction factor. These data can be acquired, for example, via the sensor 51 shown in FIG. 3.

The correction factor 54 corresponds to the correction factor f_{liquid} explained in the general part. This is dependent on sensor data 55 which can be acquired by the sensors 42 to 44 shown in FIG. 3. The sensors 42 to 44 are moisture sensors, due to which the sensor data describe the moisture in the ground 2. Because of the vertical spacing of the sensors 42 to 44, a lysimeter can also be implemented by these sensors and the vertical water flow in the ground 2 can be determined as part of the sensor data 55 and taken into consideration.

The correction factor 29 corresponds to the correction factor f_{pH} explained in the general part and depends on sensor data 34, which relate to the pH value in the ground 2 and can be acquired, for example, via the sensor 47 shown in FIG. 3.

The correction factor 30 depends on the alkalinity 19 of the ground 2, which is provided in the example shown as prior information 15. Alternatively, the alkalinity could also be sensorially acquired. The correction factor 30 therefore corresponds to the correction factor f_{Alk} explained in the general part.

Calculation rules for calculating the individual correction factors 26 to 30 from the mentioned variables are specified by the program 13. They can be determined, for example, by test series in the laboratory, wherein, for example, lookup tables, a regression analysis, statistical analyses, machine learning, and the like can be used to generate calculation rules from these experimental series.

The measured values or sensor data are preferably acquired several times a day or several times per hour by the respective measurement module 6 in order to enable a determination of the amount of the bound carbon dioxide with high time resolution. Since the relevant parameters can differ locally, for example, between different fields, a separate measurement module 6 can be used per field, as schematically shown in FIG. 1. In general, the distance 53 between measurement positions 38 to 40 or measurement modules 6 can preferably be less than 500 m.

With respect to FIG. 1, it was presumed that the energy supply device 1 is a solar cell. In order to also enable measurements at night or in bad weather, for example, it is advantageous if a battery is additionally or alternatively used as the energy supply device 48, as schematically shown in FIG. 3.

Both in the measurement module 6 shown in FIG. 1 and in the one shown in FIG. 3, it is presumed that the communication device 8 also implements the readout of the various sensors 7, 42-47, 50, 51, the control of the power supply, and under certain circumstances preprocessing of the sensor data to determine intermediate results. In principle, it is also possible to implement corresponding functions by way of a separate processing device integrated in the measurement module 6, for example, a CPU having assigned memory.

The electronic components of the measurement module can be at least partially integrated in a housing 41, which can protect the components from moisture, on the one hand, and/or prevents damage to the components, for example, during hammering into the ground or also upon being driven over, for example, by a tractor, on the other hand.

1-12. (canceled)

13. A method for determining an amount of carbon dioxide chemically bound within a time interval with the aid of a reaction agent on the ground, comprising the following steps:
reading in at least one reaction agent parameter relating to the reaction agent,
acquiring a respective measured value for the conductivity of the ground and/or for respective cationic concentration of at least one cation in the ground by way of at least one sensor attached in or on the ground, and
determining the amount of the bound carbon dioxide as a function of both the reaction agent parameter and the measured value or the measured values.

14. The method according to claim 13, wherein the amount of the bound carbon dioxide is determined as a function of sensor data of the sensor and/or at least one further sensor, wherein the sensor data relate to a vertical water flow in the ground and/or an amount of precipitation and/or a moisture in the ground and/or an alkalinity and/or a nutrient content of the ground and/or a partial pressure of carbon dioxide in the air above the ground.

15. The method according to claim 13, wherein the amount of the bound carbon dioxide is determined as a function of sensor data of the sensor and/or the further sensor and/or at least one further sensor, wherein the sensor data relate to a pH value and/or a temperature of the ground.

16. The method according to claim 13, wherein the reaction agent parameter specifies a base value for the amount of the bound carbon dioxide or for the time derivative of this amount, which is multiplied in the course of the calculation by at least one correction factor, wherein at least one of the correction factors depends on the measured value or at least one of the measured values and/or at least parts of the sensor data.

17. The method according to claim 13, wherein the concentration of sodium ions and/or of calcium ions and/or of magnesium ions is acquired as the cation concentration.

18. The method according to claim 13, wherein the respective measured value and/or the sensor data and/or at least one intermediate result determined from the measured value and/or the sensor data from a measurement position, at which the respective sensor or the respective sensors is or are arranged is wirelessly transmitted to a processing device, which carries out the determination of the amount of the bound carbon dioxide.

19. The method according to claim 13, wherein several times a day or several times per hour the measured value or the measured values and/or at least parts of the sensor data are acquired and the amount of the bound carbon dioxide is determined as a function thereof.

20. The method according to claim 13, wherein the measured value or at least one of the measured values and/or the sensor data are acquired at multiple measurement positions by a respective sensor, wherein the measurement positions are in particular spaced apart from one another by less than 500 m or less than 200 m.

21. The method according to claim 13, wherein a soil sample is taken from the ground, in particular in the area of a respective measurement position, at which the respective sensor or the respective sensors is or are arranged before acquisition of the respective measured value and/or the sensor data which soil sample, in particular spaced apart from the sample location, is studied in a laboratory in order to determine an alkalinity and/or a porosity and/or a soil type of the ground and/or fungi and/or bacteria present in the ground as prior information, wherein the amount of the bound carbon dioxide is determined as a function of the prior information.

22. The method according to claim 13, wherein a respective item of prior information, which in particular relates to an alkalinity and/or a porosity and/or a soil type of the ground and/or fungi and/or bacteria present in the ground and/or a planting of the ground and/or the weather, and/or the respective prior information determined on the basis of the soil sample is provided separately for multiple areas, wherein in a measurement area, which comprises the sensor and/or the further sensor, a position determination device is additionally used, which provides an item of position information relating to the respective measurement area, wherein on the basis of the position information, the prior information assigned to the measurement area is selected, wherein as a function of this and the at least one measured value acquired in the measurement area and/or of at least parts of the sensor data acquired in the measurement area, the amount of the bound carbon dioxide or an intermediate result, on which the determined amount of the bound carbon dioxide depends, is determined.

23. A device for determining an amount of carbon dioxide chemically bound within a time interval with the aid of a reaction agent on the ground, comprising at least one sensor for acquiring measured values for the conductivity of the ground and/or for a respective cation concentration of at least one cation in the ground and a processing device, wherein the device is configured for carrying out the method according to claim 13.

24. The device according to claim 23, wherein it comprises at least one measurement module, which comprises a housing designed to be inserted into the ground, at least one sensor, a communication device for wirelessly transmitting measured values and/or sensor data acquired by means of the sensor and/or an intermediate result determined from the measured value and/or the sensor data to the processing device and at least one energy supply device for supplying the communication device and the sensor.
